(19) United States
(12) Patent Application Publication
(10) Pub. No.: US 2024/0001068 A1
(43) Pub. Date: Jan. 4, 2024

(54) MOOD ADJUSTING METHOD AND SYSTEM BASED ON REAL-TIME BIOSENSOR SIGNALS FROM A SUBJECT

(71) Applicant: KARLSSONWILKER INC., Queens, NY (US)

(72) Inventors: Jan WILKER, Queens, NY (US); Yue ZHANG, Queens, NY (US); Dione LEE, Queens, NY (US)

(21) Appl. No.: 18/035,028

(22) PCT Filed: Nov. 17, 2021
(86) PCT No.: PCT/US2021/059688

§ 371 Date: May 2, 2023

Related U.S. Application Data
(60) Provisional application No. 63/114,806, filed on Nov. 17, 2020.

(51) Int. Cl. A61M 21/02; G16H 20/70

(52) U.S. Cl. CPC A61M 21/02; G16H 20/70; A61M 2021/0027

(57) ABSTRACT

Methods, systems, and apparatuses for adjusting a mood of a subject, the method including applying one or more sensory stimuli to a subject, obtaining one or more biosignals from the subject, the one or more biosignals being indicative or correlative of a mood of the subject, generating a stimuli signal to adjust the sensory stimuli applied to the subject, and adjusting the one or more sensory stimuli applied to the subject based on stimuli signal to obtain a desired mood in the subject.

### FIELD OF THE DISCLOSURE

The present disclosure relates to systems, apparatuses, and methods for adjusting a mood, emotion, feeling, or affective state of a subject, and particularly for adjusting a mood, emotion, feeling, or affective state of a subject based one or more sensory stimuli applied to the subject and one or more biosignals obtained from the subject.

### BACKGROUND

Sensory stimuli are known to affect or enhance the mood, emotions, feelings, and affective state of a subject to which the stimuli are applied. For example, certain auditory stimuli including sounds can affect the mood of a subject. Such auditory stimuli can, for example, cause the subject to relax or enter into a relaxed mood, causing the subject to experience a relief from stress and/or anxiety. Or alternatively, auditory stimuli can heighten senses and awareness in the subject. Auditory stimuli applied to a subject can also causes a release of dopamine or epinephrine, lower or increase amounts of cortisol, and causes other changes in levels of hormone and neural transmitters in the subject, causing different feelings of relaxation, euphoria, cravings, and excitement. For this reason, auditory stimuli, such as music, is known to have a significant effect on the mood, emotions, feelings, and affective state of a subject to which the stimuli are applied. This is evidenced by the relaxation felt by some while listening to classical or instrumental music. Or alternatively, by the excitement felt by a player listening to upbeat music while preparing mentally for a physical contest, such as American football, or by the motivating effect of appropriate music during performance of difficult physical activities, such as weight lifting or long-distance running, or by the euphoria felt by attendees at a rock concert that engage in headbanging behavior while listening to and feeling the vibrations of rock, punk, or heavy metal music genres.

Additionally, the effect of the applied sensory stimuli are known to have a measurable effect on a subject. For example, a release of hormones and neural transmitters, caused, for example, by auditory stimuli, causes measurable physiological or psychological changes in the subject. Such physiological or psychological changes may include changes in heart rate, blood pressure, body temperature, blood oxygen saturation, and sweating.

Similar to auditory stimuli, other sensory stimuli, such as visual, tactile, olfactory, and taste-based stimuli are known to causes measurable physiological or psychological effects in a subject. For this reason, in the field of massage therapy it is popular to provide lighting in a room that can cause the occupants to feel relaxed. And olfactory-based stimuli are known to have significant measurable physiological or psychological effects in a subject. For example, a foul odor can cause the subject to become physically ill, while a pleasant odor causes a subject to relax. In medical applications, smelling salts are known to arouse consciousness.

Although sensory stimuli, such as auditory stimuli, are known to affect the mood, emotions, feelings, and affective state of a subject to which the stimuli are applied, what the inventors of the present application have identified as a significant problem is that the efficiency and speed in causing a predetermined and desired change in mood, emotions, feelings, and affective state has not been well understood or studied. For example, treatment centers have been known where auditory stimuli have been applied to a subject and certain biofeedback signals have been obtained attempting to measure the effect of the applied auditory stimuli.

Additionally, certain computer applications (apps) have been developed for use by consumers, such as Calm, Headspace, Waking Up, and other meditation/mindfulness apps, that have been developed in what has been termed the self-care industry for stress relief and wellness. Such treatment centers and apps have been found to relieve stress and increase wellness. But as noted above, the efficiency of these programs and apps could be greatly improved. With growing public attention on mental health and how to avoid the harmful effects of too much stress, the demand for effective solutions is increasing, particularly with the limited amount of time and money subjects are able to spend on selfcare.

It is further noted that the need for efficient and effective selfcare, such as producing relaxation in the subject, is rising. For example, a 2019 Gallup World Poll found that people in the United States experienced 25% more stress, 32% more worry, and 38% more anger in 2018 than they had experienced in 2008. Additionally, the use of consumer applications is on the rise. For example, Headspace has been found to have 31 million users, with more than 1 million “premium” members of the provided service. Similarly, Calm has 26 million users, with more than 1 million “premium” members. Calm was nominated by Apple's App Store editors as one of their top apps of 2017, and Calm adds 50,000 new users each day. Further, with the COVID-19 pandemic of 2020, and the loss of loved ones, family, and friends, and the required social distancing, resultant worldwide economic downfall, and the closing of businesses, restaurants, and schools, the amount of stress, worry, and anxiety felt by the population has increased significantly.

### SUMMARY

Methods, systems, and apparatuses for adjusting a mood of a subject are provided, at least one method including applying one or more sensory stimuli to a subject, obtaining one or more biosignals from the subject, the one or more biosignals being indicative or correlative of a mood of the subject, generating a stimuli signal to adjust the sensory stimuli applied to the subject, and adjusting the one or more sensory stimuli applied to the subject based on stimuli signal to obtain a desired mood in the subject.

A system is provided for adjusting a mood of a subject, the system comprising a sensory stimulator system configured to apply one or more sensory stimuli to a subject, a sensor system configured to obtain one or more biosignals from the subject, the one or more biosignals being indicative or correlative of a mood of the subject, and a computer system having one or more processors configured to receive the one or more obtained biosignals, and based thereon, generate a stimuli signal to adjust the sensory stimuli applied to the subject by the sensory stimulator system. The sensory stimulator system adjusts the one or more sensory stimuli applied to the subject based on the generated stimuli signal to obtain a predetermined mood, emotion, feeling, or affective state in the subject.

A hardware storage device is provided having stored thereon computer executable instructions which, when executed by one or more processors of a computer system, configure the computer system to perform at least the following: apply one or more sensory stimuli to a subject; obtain one or more biosignals from the subject, the one or more biosignals being indicative or correlative of a mood of the subject; receive the one or more obtained biosignals and processing said biosignals by a computer system having one or more processors and based thereon, generating a stimuli signal to adjust the sensory stimuli applied to the subject; and adjust the one or more sensory stimuli applied to the subject based on the generated stimuli signal to obtain a predetermined mood, emotion, feeling, or affective state in the subject.

### BRIEF DESCRIPTION OF THE DRAWINGS

FIG. 1 shows a schematic diagram of a mood-adjusting system according to an embodiment of the disclosure.

FIG. 2 shows a schematic diagram of a mood-adjusting system according to another embodiment of the disclosure.

FIG. 3 shows a schematic diagram of a mood-adjusting system according to another embodiment of the disclosure.

FIG. 4A shows a schematic diagram of a mood-adjusting system according to another embodiment of the disclosure.

FIG. 4B shows a schematic diagram of a mood-adjusting system according to another embodiment of the disclosure.

FIG. 5 shows a schematic diagram of a mood-adjusting system according to another embodiment of the disclosure.

FIG. 6 shows a sequence of soundscapes in modular design for a mood-adjusting system and method according to another embodiment of the disclosure.

FIG. 7 shows test protocols of soundscapes for a mood-adjusting system and method according to another embodiment of the disclosure.

FIG. 8 shows test results obtained by a mood-adjusting system and method as described herein.

FIG. 9 shows an embodiment of an experience booth according to another embodiment of the disclosure.

FIG. 10A shows a display of a mobile platform of an embodiment the mood-adjusting system according to another embodiment of the disclosure.

FIG. 10B shows a display of a mobile platform of an embodiment the mood-adjusting system according to another embodiment of the disclosure.

FIG. 11 shows a hardware diagnostic of an embodiment a mood-adjusting system according to another embodiment of the disclosure.

FIG. 12A shows a schematic diagram of a mood-adjusting system according to another embodiment of the disclosure.

FIG. 12B shows a schematic diagram of a mood-adjusting system according to another embodiment of the disclosure.

FIG. 13 shows a schematic diagram of a mood-adjusting system according to another embodiment of the disclosure.

FIG. 14 shows a schematic diagram of a mood-adjusting system according to another embodiment of the disclosure.

FIG. 15 s shows a schematic diagram of a mood-adjusting system according to another embodiment of the disclosure.

FIG. 16 shows a schematic diagram of a mood-adjusting system according to another embodiment of the disclosure.

FIG. 17 shows a schematic diagram of soundscape production system according to an embodiment of a mood-adjusting system according to another embodiment of the disclosure.

FIG. 18 shows a schematic diagram of steps of a mood-adjusting method according to another embodiment of the disclosure.

FIG. 19A, FIG. 19B, and FIG. 19C show an icon on a display of a mobile device according to an embodiment of a mood-adjusting system according to another embodiment of the disclosure.

FIG. 20A and FIG. 20B show an icon on a display of a mobile device according to an embodiment of a mood-adjusting system according to another embodiment of the disclosure.

FIGS. 21A, 21B, 21C, 21D, 21E, 21F, 21G, 21H, 21I, 21J, 21K, and 21L show various icon on a display of a mobile device according to an embodiment of a mood-adjusting system according to another embodiment of the disclosure.

FIG. 22A, FIG. 22B, FIG. 22C, and FIG. 22D show examples of biosensor data obtained and decision events used in the generation of soundscape signals to adjust the mood of the subject according to another embodiment of the disclosure.

FIG. 23 shows variables considered in the generation of soundscape signals to adjust the mood of the subject according to another embodiment of the disclosure.

The drawing figures are not drawn to scale, but instead are drawn to provide a better understanding of the components and are not intended to be limiting in scope, but to provide exemplary illustrations.

### DETAILED DESCRIPTION OF VARIOUS EMBODIMENTS

While the disclosure is susceptible to various modifications and alternative constructions, certain illustrative embodiments are in the drawings described below. It should be understood, however, there is no intention to limit the disclosure to the specific embodiments disclosed, but on the contrary, the intention covers all modifications, alternative constructions, combinations, and equivalents falling within the spirit and scope of the disclosure.

A better understanding of the disclosure's different embodiments may be had from the following description read with the accompanying drawings in which like reference characters refer to like elements.

For an understanding of the interface system of the present disclosure, reference is made such relaxing platforms such as Calm or Headspace, mentioned above. These have promised to relieve stress and produce relaxation in the subject. Subject using these platforms choose soundscapes, music, and audio programming through a manual process hoping that the resultant auditory experience will bring calm to them. But the actual physical responses of the subject are not measured and are not considered. Alternatives such as therapy and meditation classes are not as accessible, due to pricing and availability.

The problems with existing and known mood adjusting platforms, such as the relaxations platforms of Calm and Headspace are at least as follows. First, these platforms have an undesirable learning curve. They take time for the user to figure out which aspects of the platform work for his or her condition or state and at what times. Platforms do not automatically tune to the right settings. Second, they require laborious production. The known mood-adjusting methods and systems often require a money payment per episode produced. Whether it's guided meditation or celebrity-voiced story, new content must be continuously added to the library to maintain audience engagement. And third, known and existing relation systems often provide generic content. Current audio relaxation platforms are often pre-recorded soundscapes hoping for a positive effect on the user, appealing to the widest audience without attention to users' immediate, actual reaction. Such methods therefore often take the one-size-fits-all approach, which produces at best mediocre results and are not as efficient or effective as could be for varying users.

In view of the above, the inventors of the present application have identified the problem that the efficiency and overall effect on the subject through application of sensory stimuli can be improved significantly from what is current done or known. Such a development would create a significant opportunity and improvement from known systems and methods, to more efficiently, effectively, and lastingly alter the mood, emotions, feelings, or affective state of the subject through measured application of sensory stimuli while obtaining measured biosignals from the subject, and based on the measured signals, adjusting the applied sensory stimuli. Additionally, based on the unique physiological, psychological, and personality characteristics of the subject, once the preferred sensory stimuli are determined (including a determined sequence of stimuli) such information can be recorded and subsequently used during later treatment sessions.

Accordingly, the aim and object of the present disclosure is to surpass the original treatment experience above through automated tuning using real-time biosensors in conjunction with artificial intelligence and machine learning as used by a computer system to improve treatment.

Although much of the present disclosure is directed to causing measurable relaxation in the subject, as noted above, similar principles and embodiments could be applied to cause different moods, feelings, emotions, of affective states in the subject, including euphoria, excitement, anxiety, cravings, motivation, etc. Additionally, while the subjects in the embodiments are shown to be human subjects, similar inventive principles could be applied to non-human subjects, including animals, such as dogs, cats, horses or other livestock in which a determined change in mood, feelings, emotions, or affective state is desired.

Relating to a treatment to increase relaxation felt by a subject, the process may start by laying the subject onto a small bed in a chamber with minimal sensory inputs. For example, the room may be darkened and the walls of the chamber may be soundproof. The only source of input to the subject is a high-fidelity sound system. The subject is first connected to a vital-signs monitor. A computer system controls the sound signals being sent to the chamber and receives and processes the vital signs response. Based on the received vital signs, the computer may adjust the settings of the sound to relax the subject. This is reflected in a change in the vital signs reading, such as a decreased heart rate or respiration rate. As such the computer system can efficiently and effectively induce a state of deep relaxation and sleep for the subject within minutes. Each session may be, for example, 30 minutes and at the end of the session the computer system will generate sounds that rouse the subject. At the end of this treatment the subject is fully relaxed and rejuvenated.

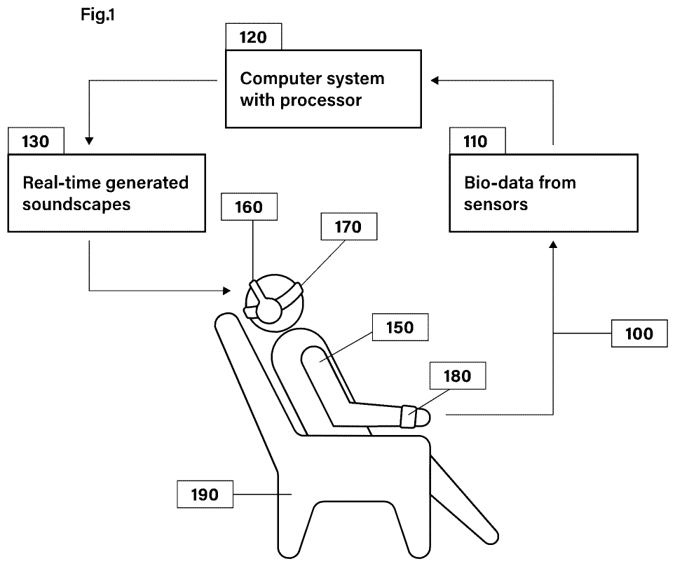

A first embodiment of such a system is shown in FIG. 1, which schematically shows a relaxation-providing system 100 The system 100 includes a computer-based processing system 120, bio sensors 170 and 180, which may include temperature sensors, pulse oximetry, and electrodermal activity (EDA) to obtain biosignals from the subject 150. Subject 150 may be positioned in a relaxed position such as in a comfortable chair 190. Bio-data 110 is obtained from the biosensors 170 and 180 and is transmitted, for example, through hard wiring or a network system such as Bluetooth or a Wide area network (WAN) to computer system 120. Computer system 120 receives the bio-data from the subject, and based thereon, provides signals for generation of auditory stimuli to produce real-time generated soundscapes 130, and speaker system 160 produces and applies the soundscape based on soundscape data 130 provided by the computer system 120.

Accordingly, the system 100 of the embodiment of FIG. 1 is based on a real-time biofeedback system 100 including the computer system 120 that may provide machine-based learning and cloud-based infrastructure. Such a system 100, based on artificial intelligence (AI) provides surpassing relaxation treatment experience through automated tuning of soundscape settings through determined algorithms guided by biofeedback sensors to efficiently and effectively deliver a personalized relaxation experience each and every time. Such a system can also learn to adjust the application of such an experience based on known variables, such as time of day, weather, days of the week, outside temperature, etc. Such a system is therefore capable of learning from previous sessions and improving and deepening experience for each and every user. The system 100 of the embodiment of FIG. 1 thus efficiently and effectively provides vitalization and mind rejuvenation to the subject, reduced stress, and may provide sleep improvement.

Various biosensors are described and provided in the embodiments described herein. However, it is noted that the biosensors should not be limited to the specific sensors described herein, but rather the significance of the sensor is to obtain biosignal data related to the desired adjustment in mood, emotions, feelings, or affective state of the subject. Such sensors therefore may include, but are not limited to, a sensor that obtains one or more biosignals obtained from the subject include data relating to electrodermal activity (EDA), galvanic skin response (GSR), electrodermal response (EDR), psychogalvanic reflex (PGR), skin conductance response (SCR), sympathetic skin response (SSR) and skin conductance level (SCL), blood pressure (BP), pulse oximetry, oxygen saturation, electroencephalography (EEG), electromyography (EMG), body movement based on one or more accelerometers or one or more gyroscopes, electrocardiography (ECG), temperature of the subject, thermal imaging, respiration, visual images of the subject, heart rate (HR), heart rate variability (HRV), photoelectric plethysmography (PPG), photoplethysmography imaging (PPGI), prefrontal cortex activity, oxyhemoglobin (oxy-Hb) concentration, cortisol levels including salivary cortisol levels, hair cortisol levels, and/or fingernail cortisol levels, pupil dilation, pupillometry, pulsimetry, accelerated plethysmography (APG), optical imaging of tissues of the subject including functional near infrared spectroscopy (fNIRS), functional magnetic resonance imaging (fMRI), computed tomography (CT), magnetoencephalography (MEG), positron emission tomography (PET), or infrared spectroscopy (NIRS).

For example, galvanic skin response (GSR) is based on a the measured skin resistance due to the sweat glands of the skin. Sweating is controlled by the sympathetic nervous system, and skin conductance is an indication of psychological or physiological arousal. It is understood that if the sympathetic branch of the autonomic nervous system becomes aroused, sweat gland activity increases, which in turn increases skin conductance. In this way, skin conductance can be a measure of emotional and sympathetic responses, and reduced skin conductance is correlated with a relaxation of the subject.

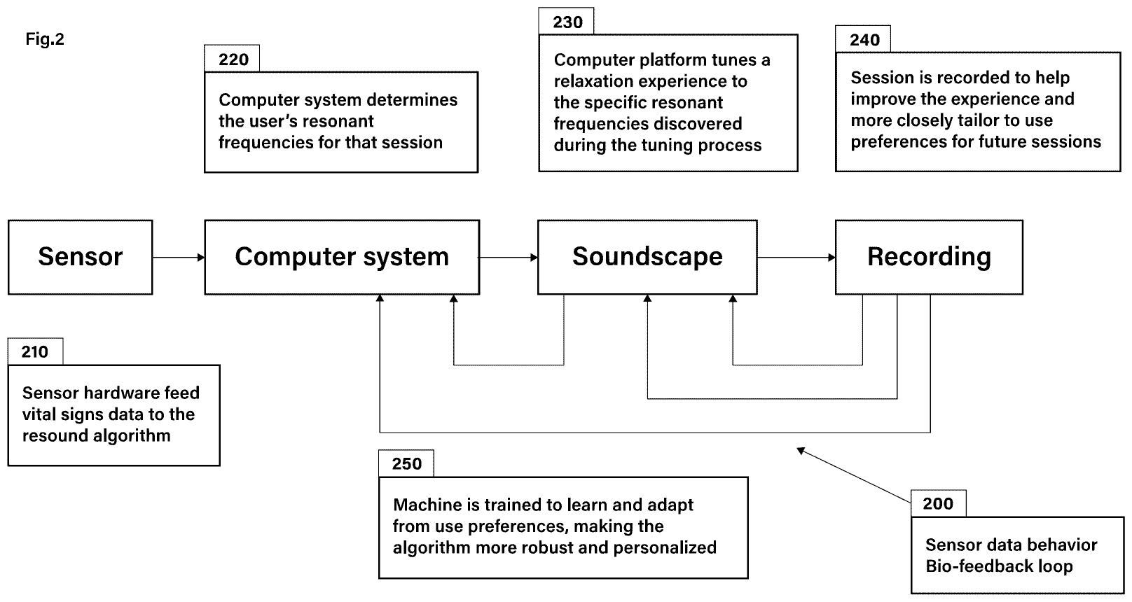

FIG. 2 shows a schematic view of a mood-adjusting system 200 based on a computer system that receives biosignal data from the subject through a sensor. At 210 the sensor hardware feeds vital signs data, or in other words, biosignal data, to the computer system. At 220 the computer system determines the subject's resonant frequencies for that session. At 230 the computer platform tunes a relaxation experience to the specific frequencies to the specific resonant frequencies discovered during the tuning processing. At 240 the session is recorded to help improve the experience and more closely tailor to use preferences for future sessions. The computer system is trained at 250 to learn and adapted from user preferences of the subject, making an applied algorithm more robust and personalized.

A soundscape generally is considered to mean a sound or combination of sounds that forms or arises from an immersive environment. The term may refer to both the natural acoustic environment, including natural sounds, biophony, the sounds of weather and other natural elements, environmental sounds created by humans, such as musical composition, sound design, and language, work, and sounds of mechanical origin resulting from use of industrial technology. Crucially, the term soundscape also includes the listener's perception of sounds heard as an environment.

In the context of this disclosure, the term “soundscape” is intended to refer to an audio signal or recording or performance of sounds that create the perceived sensation of a particular acoustic environment, or compositions created using the sounds of an acoustic environment, either exclusively or in conjunction with musical performances. A generated soundscape may include various auditory signals or combinations of auditory signals with predetermined and/or varying frequencies, volumes, timbers, harmonies or harmonics, beats, rhythms, and/or binaural beats, etc.

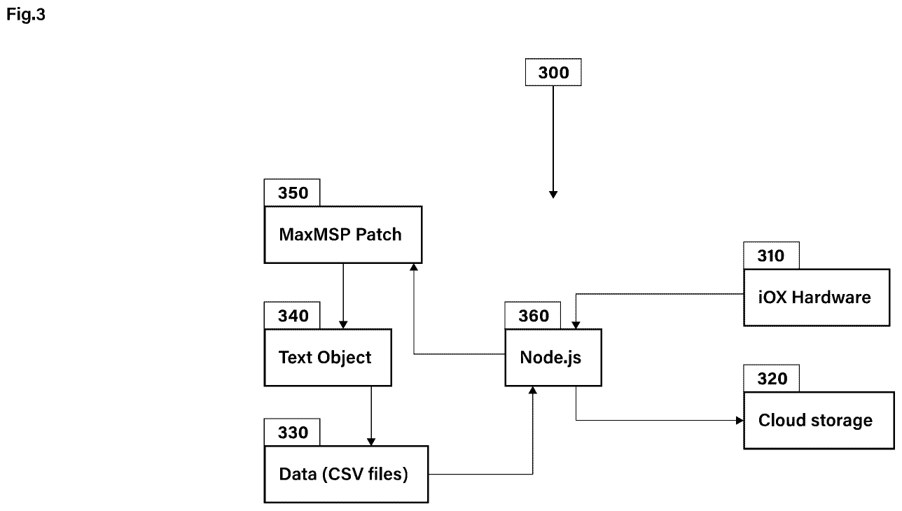

The embodiment of FIG. 3 shows a mood-adjusting system 300 showing a manner in which data is exported a visual explanation of how the data is integrated and passes into each component. In the embodiment of FIG. 3, MaxMSP may serve as the soundscape generation platform. NodJS may be the communication protocol to the cloud and to MaxMSP. MySQL is an example of an online database storage for user data received from the subject. React Natvie is the front-end interface for Android and iOS phones, which may serve as the computer system. And the biosensors and biofeedback hardware may include a GSR sensor, a microcontroller board (Arduino), a pulse oximeter, and an Apple watch or other smart watch devices. In this case, a sensor, such as an iOximeter 310 is attached to the person and an app is updated to connect via NodeJS 360 to a MaxMSP part file 350. A text Object 340 is used to export both the soundscape settings as well as the data from the iOximeter 310. The data is exported and saved as a CSV file 330. The NodeJS 360 connection takes the data from CSV file 330 and uploads it to the Cloud storage 320. The Cloud storage 320 may store all recorded sessions from every session in every location.

It should be noted that although MaxMSP is described in the embodiment above, the disclosure itself of course should not be considered to be limited to a MaxMSP platform or language. Other platforms or languages may equivalently be used, including but not limited to Pure Data (PD), AudioMulch, Bidule, Kyma, TouchDesigner, vvvv, OpenMusic, Nodal, and or other visual programming platforms.

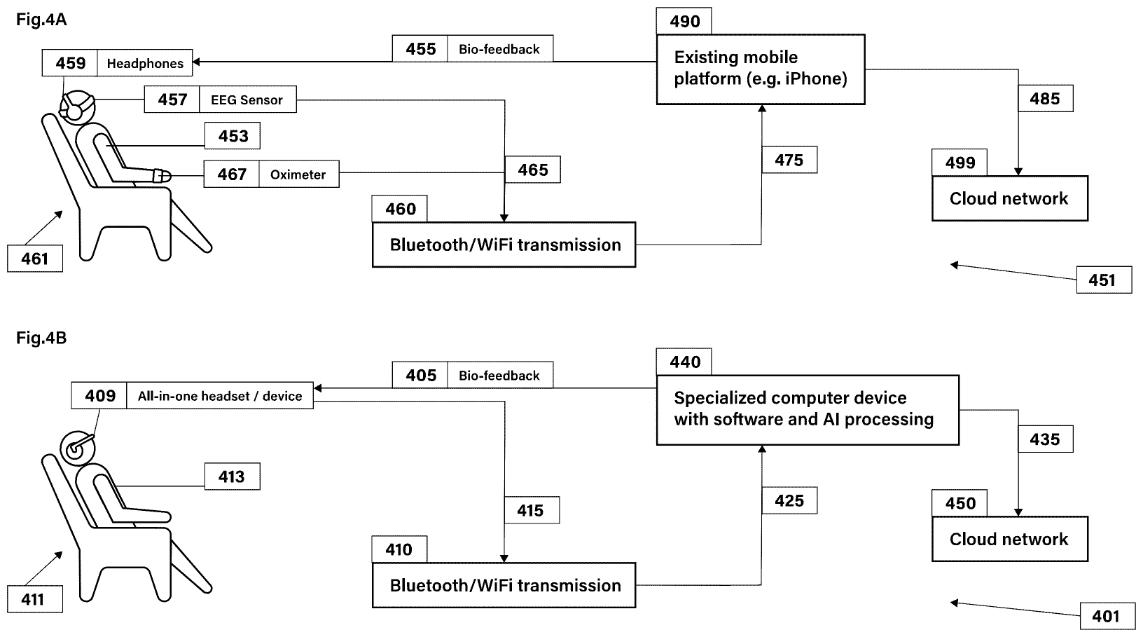

FIGS. 4A and 4B show different embodiments of a mood-adjusting system 401, 451. In system 451 (illustrated in FIG. 4A), a subject 453 reclines comfortably, for example, in chair 461. Subject 453 is provided with various sensors including, but not limited to, an electroencephalography (EEG) sensor 457 and oximeter 467. High-fidelity headphones 459 are placed over the ears of the subject. Biofeedback from sensors, such as EEG sensor 457 and oximeter 467 is generated and transmitted 465 by the sensors, either through hard wires or a wireless connection, such as by a Bluetooth/WiFi transmission 460, and transmitted at 475 to an existing mobile platform 490. Based on the biosignals, the platform 490 produces a biofeedback-based soundscape signal 455 that is received by headphones 459 that produces the desired adjustments to the audio signal applied to the subject 453. Additionally, the biofeedback and soundscape signals are further transmitted 485 to the cloud network 499 to be stored and used in future mood-altering processing. In this embodiment, processing of the biosensor data may be performed by the existing mobile platform 490. Or alternatively, the existing mobile platform may transmit the biosensor data to a server or processor within the cloud network, and appropriate sound signals may be transmitted through the network to the mobile platform 490 and from there, transmitted to the headphones 459 worn by the subject.

In the embodiment of FIG. 4A, is advantageous in that it is developed around an existing platforms (e.g., an iPhone) and biodata sensors, for ease of access and to gather as much data as possible. This allows the machine learning and data processing, for example, an AI algorithm, to be developed and refined more efficiently. Users will be able to use most of their own devices as long as they have the app.

The mood-adjusting system 401 of FIG. 4B is similar to that of the embodiment of FIG. 4A, but differs in that one or more sensors, such as an EEG sensor or blood oximeter, or a galvanic skin response (GSR) are combined with high-fidelity headphones in an all-in-one headset device 409 worn by user 413, while relaxing in chair 411. Biofeedback from the sensors of headset device 409 is generated and transmitted 415 by the sensors, either through hard wires or a wireless connection, such as by a Bluetooth/WiFi transmission 410, and transmitted at 425 to a specialized computer device 440 with software and AI processing. Based on the biosignals, the specialized computer device 440 produces a biofeedback-based soundscape signal 405 that is received by headset device 409 that produces the desired adjustments to the audio signal applied to the subject 413. Additionally, the biofeedback and soundscape signals may be further transmitted 435 to the cloud network 450 to be stored and used in future mood-altering processing.

The embodiment of FIG. 4B introduces specialized hardware that condenses all the processes and sensors involved into one sleek and efficient product and system. This generation will utilize data collected from earlier sessions to aid in the machine learning, and provide state-of-the-art technology to create the best possible user experience.

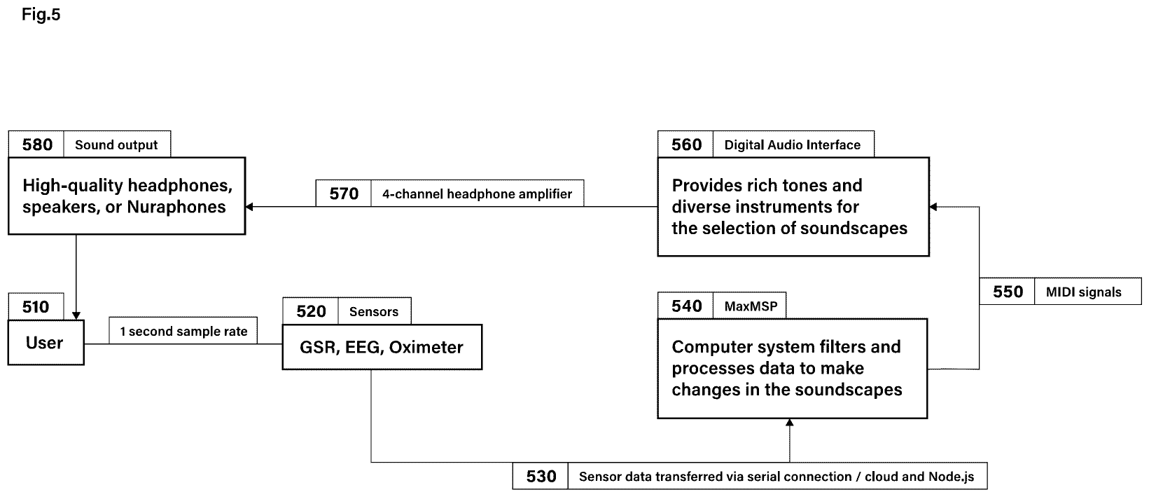

In the embodiment of the system 500 shown in FIG. 5, biosignals are obtained form user 510, for example, as a 1-second sample rate, by sensors 520 (e.g. GSR, EEG, oximeter). Preferably, the sample rate is higher than a 1-second sample rate, such as a sample rate of 0.1 second (10 Htz) or even a sample of rate of 0.01 seconds (100 Htz). But if needed, due to processing limitations in some embodiments, lower sample rates may also be preferred, such as a sample rate (0.1 Htz). The biosignals are transmitted to a computer system 540 with MaxMSP, which filters and processes data to make changes in a soundscape to be applied. The sensor data maybe transferred to MaxMSP via serial connection/cloud and NodeJS 530. MIDI signals 550 are transmitted to a digital audio interface 560, which provides rich tones and diverse instruments of the selection of soundscapes. A 4-channel headphone amplifier 570 is provided to a sound output 580, which includes high-quality headphones, speakers, or nuraphones, which then applies the soundscape auditory signal to the subject.

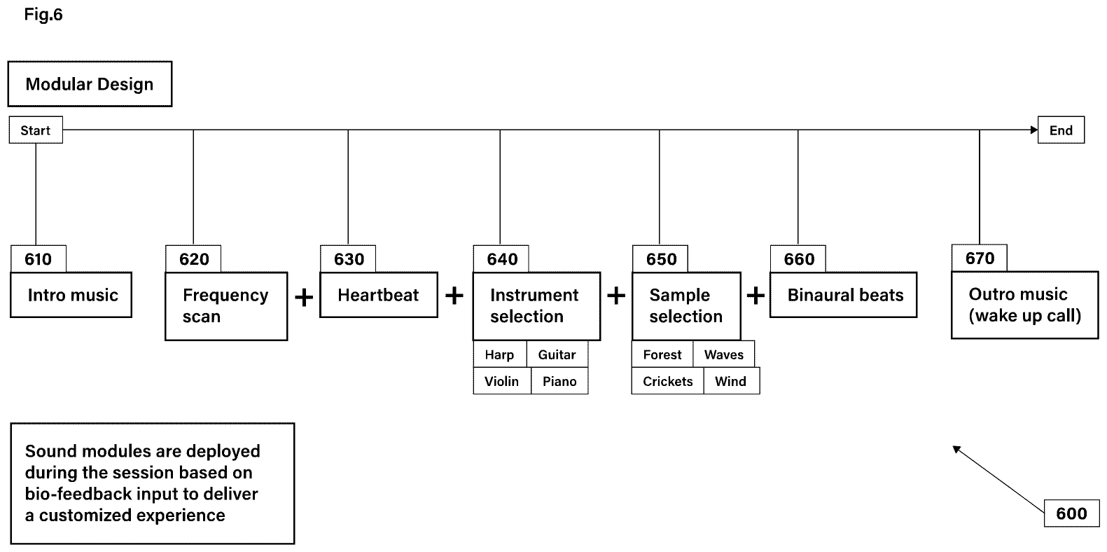

As shown in FIG. 6, the applied auditory signal may include a sequence of soundscapes in modular design 600. Such a sequence of sound modules may include: intro music 610, a frequency scan or varying frequencies 620, a rhythm 630, such as a drum or mimicked heartbeat, an instrument selection 640 including, for example, a harp, acoustic guitar, or electric guitar, a sample selection of natural sounds 650, such as crickets or plants, or other biophany, or natural sounds such as rain, ocean waves, thunder, etc., and binaural beats 660. The session may be closed with outro music 670 that serves as a wake-up call. Although shown in sequence, the above components of the modular design may be planed consecutively as shown or not consecutively but rather in a different order than shown, or may be applied to the subject concurrently, such as binaural beats 660 applied during or in the background of the sample selection of natural sound 650 or musical instrument selection 640, or both. The sound modules are deployed during the session based on biofeedback input to deliver a customer experience to the subject in real time based on the measured biosignal data received from the subject.

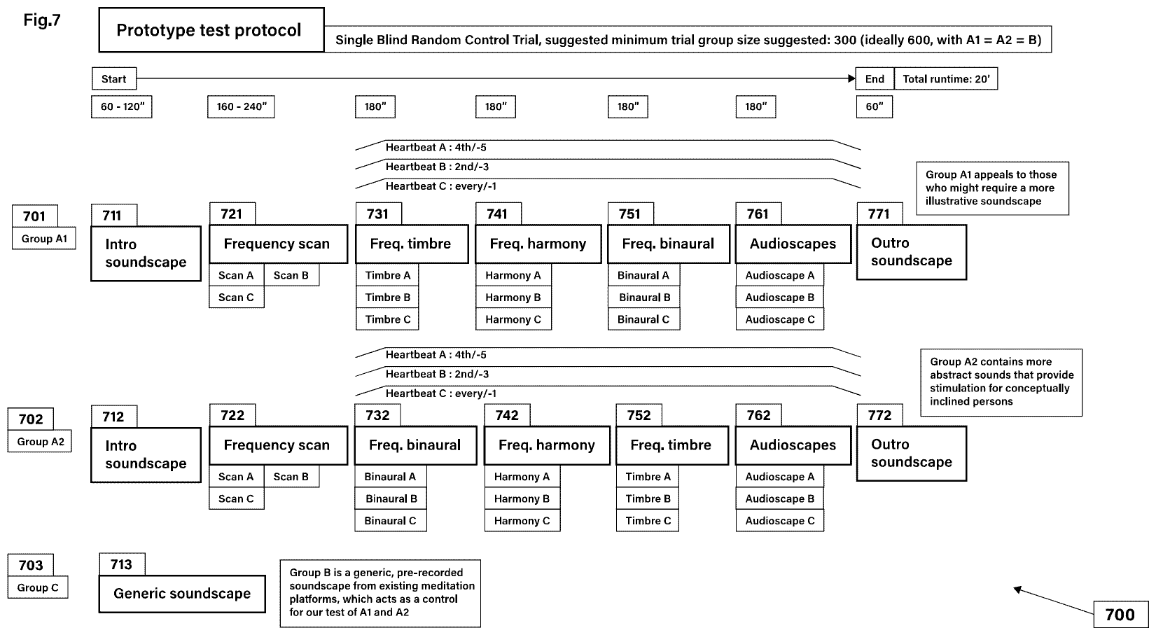

The embodiments of FIG. 7 include various prototype test protocols 700, including test protocol A1 (701), test protocol A2 (702), and control protocol B (703). Protocol A1 (701) may appeal to those who might require a more illustrative soundscape, and includes sound block 711 of 60-120 seconds of an intro soundscape; sound block 721 of 180-240 seconds of frequency scans A, B, and C; sound block 731 of 180 seconds with different frequencies with different timbres A, B, and C; sound block 741 for 180 seconds with frequencies and different harmonies A, B, and C; sound block 751 for 180 seconds with frequencies with binaural beats A, B, and C; sound block 761 with soundscape A, B, and C; and sound block 771 with an outro soundscape. Additionally, heartbeats A, B, and C may be applied concurrently throughout soundblocks 731, 741, 751, and 761.

Protocol A2 (702) contains more abstract sounds that provide stimulation for conceptually-inclined persons and may appeal to those who might require a more illustrative soundscape. Protocol A2 (702) includes sound block 712 of 60-120 seconds of an intro soundscape; sound block 722 of 180-240 seconds of frequency scans A, B, and C; sound block 732 for 180 seconds with frequencies with binaural beats A, B, and C; sound block 742 for 180 seconds with frequencies and different harmonies A, B, and C; sound block 752 of 180 seconds with different frequencies with different timbres A, B, and C; sound block 762 with soundscape A, B, and C; and sound block 772 with an outro soundscape. As a control in testing, protocol B (703) may be a generic soundscape, for example as provided by Headspace, Calm, Omvana, or RelaxMelodies, etc., applied to the subject over the entire sound block 713.

As each of Protocols A1, A2, and B are applied to the subject, biosignal data is obtained to determine the physiological or psychological response of the subject, and to determine which soundscape most effectively produces the desired physiological or psychological response or physiological or psychological changes in the subject, based for example, on GSR, EEG, pulse oximetry, etc. It should be noted that although examples protocols are provided in FIG. 7 with particular sequences. The sequences of the test protocol should not be limited to the sound block sequences provided therein, but rather the sound block sequences may be varied in a different order, may include additional sound blocks, or the time of the sound blocks may be varied.

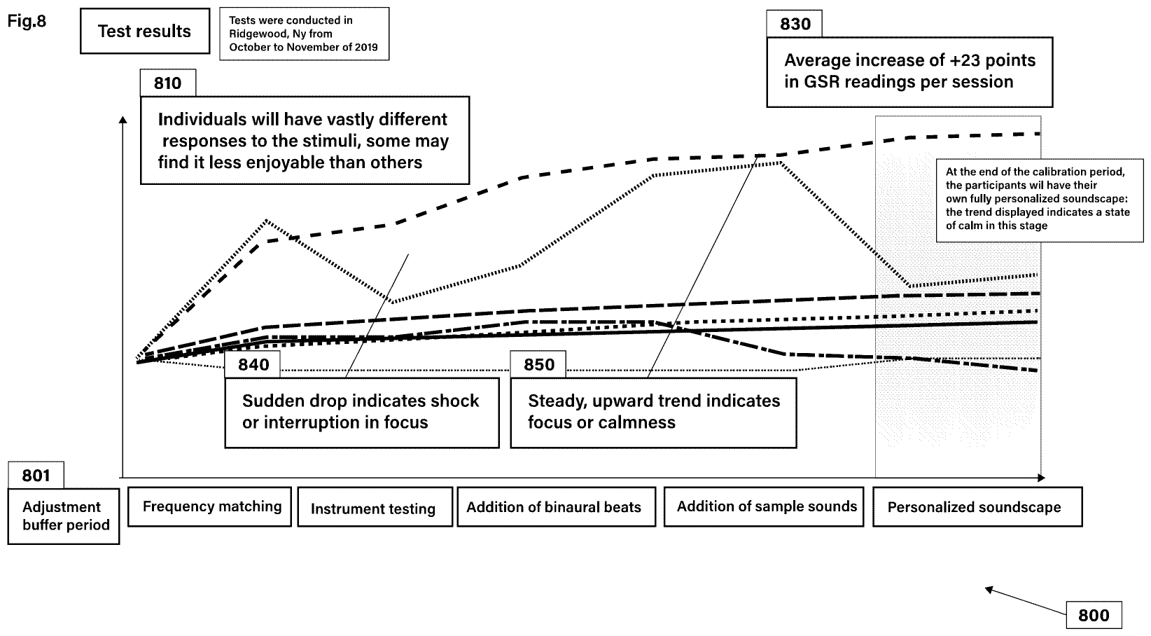

FIG. 8 shows test results 800 of the inventors' system based on tests conducted on various subjects in Ridgewood, NY. It was found that individuals have vastly different responses to the auditory stimuli 810. Some subjects find some test protocols more enjoyable than others. During the tests, GSR biosignals were obtained from the subjects. And an average increase of +23 points in GSR readings per session were obtained as compared to the control protocol B (703). FIG. 8 shows GSR readings from various subjects tested in relation to time elapsed with an adjustment buffer prior 801. During frequency matching, sudden drops in GSR readings indicate a shock or interruption in focus 840. And a steady, upward trend indicates focus or calmness 850. At the end of the calibration periods, the test subjects were able to obtain their own fully personalized soundscape: the trend displayed indicates a state of calm in this stage.

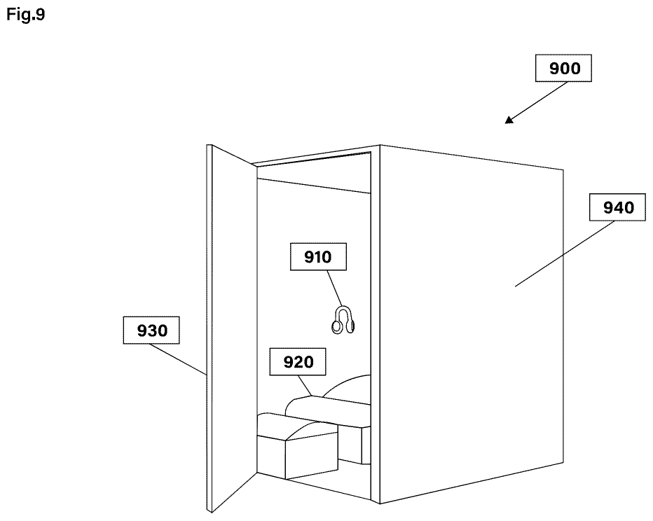

FIG. 9 shows an embodiment of an experience booth 900 that may be used in conjunction with the disclosed embodiments of the mood-adjusting systems and methods. Experience booth 900 may be similar to a telephone booth with soundproof walls 940 and door 930. Noise-isolating foam and structures can surround the user and eliminate ambient sounds. A subwoofer can also be attached to the listening chair to add maximum physical effect. The booth may have soundproof walls 940 and door 930. Within the experience booth 900, the subject is provided with a comfortable chair or bed 920 and high-fidelity headphones 910.

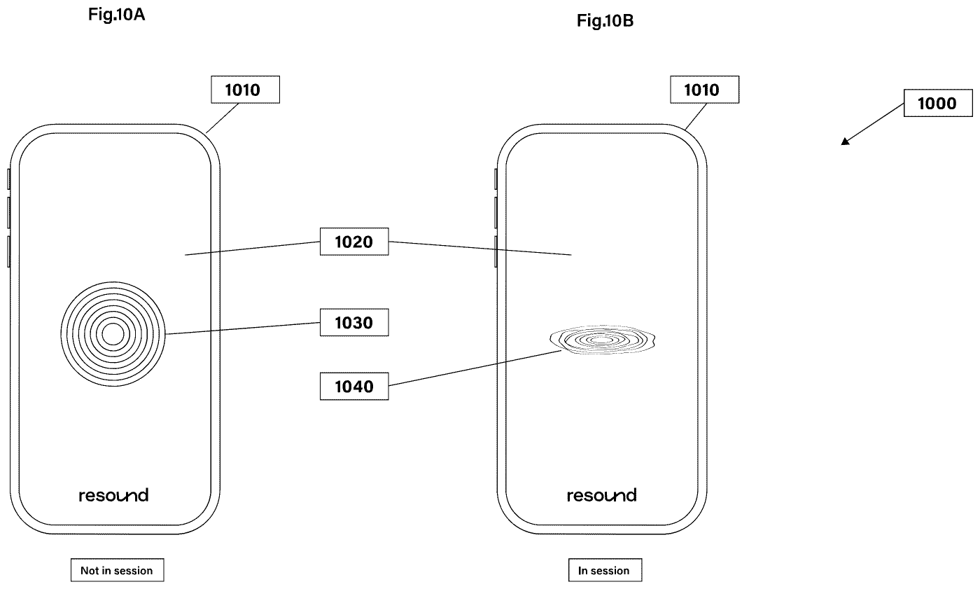

In the embodiments described herein, processing of the biosensor data may be performed and generation of the adjusted soundscape signals may be performed by the processor of an existing mobile platform 1010, such as a mobile phone or iPhone owned by the subject, on which an app is provided. Or alternatively, the existing mobile platform 1010, rather than process the biosensor data and generate the soundscape signals, may transmit the biosensor data to a server or processor within a cloud network, and appropriate soundscape signals may be generated and transmitted through the network to the mobile platform 1010 and from there, transmitted to, for example, headphones worn by the subject. FIGS. 10A and 10B show a mobile system 1000 including mobile platforms 1010 with displays 1020 when the mood-adjusting app is not in session 1030 and when the app is in session 1040. Such an app arrangement on the mobile platform 1010 of the subject provides an easy to use system with a simple interface that reduces tweaking and distracting features on the mobile phone. Through the mobile phone, system is able to processing the biosensor signals and self-adjust to provide an effective and efficient adjustment in the mood of the subject.

In conjunction with the mobile platforms 1010 of FIGS. 10A and 10B, FIG. 11 shows hardware diagnostics 1100 may be obtained through, for example, a smart watch 1110 worn by the subject with wristband 1150, to obtain the biosignals. Such smart watches may include an Apple Watch, Samsung Great, or a Fitbit Versa 2. The smart watch 1110 may have a display 1120 showing an app icon 1140 similar to that as shown in FIG. 10B when in session. Other hardware diagnostics may also include an electroencephalogram, such as a Muse Headband, a galvanic skin response sensor, a SafeHeart iOx pulse Oximeter, or other hardware.

According to other embodiments, an integrated system would include a vital signs monitor such as a Safe Heart iOx pulse oximeter that would feed the vital signs data as the biosignal data directly into a device that is termed here as a “ReSound box”, which is driver device for the speakers. The ReSound box would include a box that is connected to the internet that downloads high quality 256-bit original digital recordings from the cloud. The box is constructed with a high quality DAC (Digital to Analog Converter) and precisely balanced headphones that may include noise cancellation to ensure a consistent experience across all users. The dimensions of the box may be no bigger than the original iPod.

To use, the user subject places the headphones on their head and clips the vital signs monitor onto one of their fingers. On the app side, the user only needs to set one setting—session duration—whether to have a quick or longer session. Upon clicking a <Start Session> button, the Resound box would, based on the duration setting, begin an audio countdown to the session starting. The auditory guide would offer suggestions for body position and breathing, and also suggest possibly an eye mask to block out ambient light. In the first session, the ReSound box would play some test tones to establish a baseline and measure the physiological and psychological response of the user subject through the measured data. Using computer system having a processor configured to performed machine learning algorithms, the tones, volumes, frequencies, would be adjusted to suit the individual. After the first calibrating session is complete, the device would then continuously self-adjust to deliver more effective sessions over time.

According to another embodiment, Safe Heart provides the mobile vital signs monitors for use. Audio files are played back using a custom computer program through headphones or an audio setup plugged into the computer system. The computer system may be tied directly with the Safe Heart data cloud to stream the live readings, but such an arrangement is not necessary, as long as the vital signs with the biosignal data are able to be obtained.

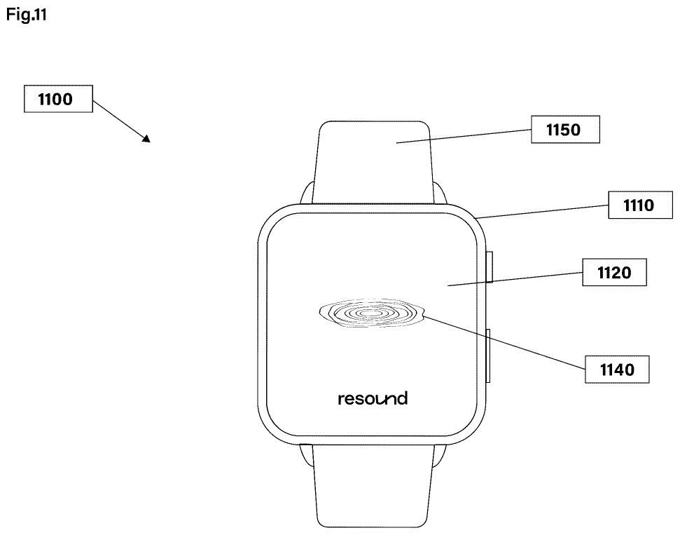

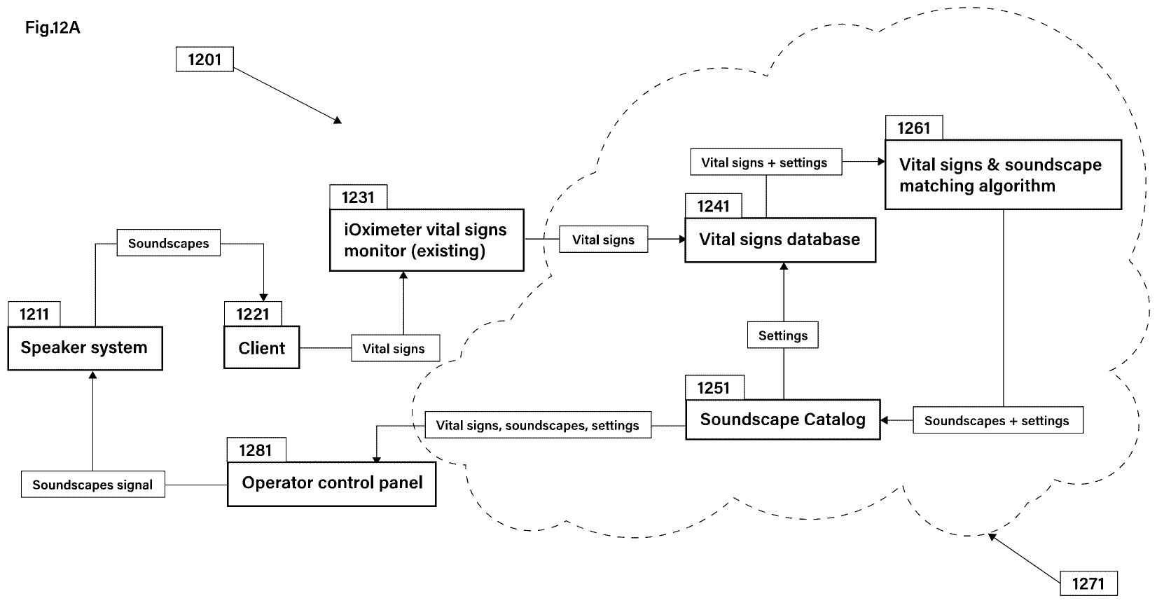

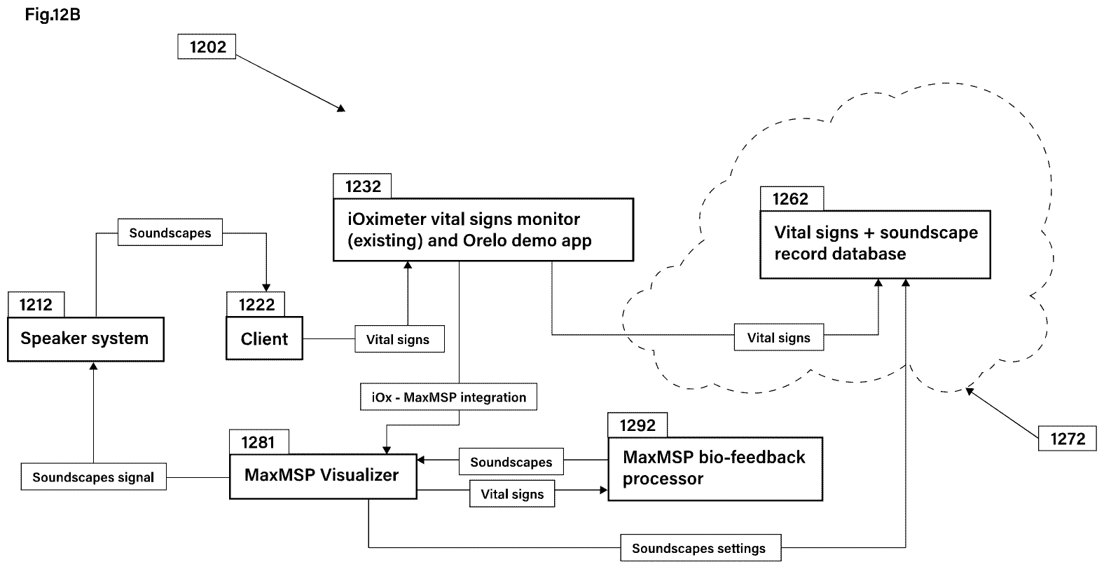

FIG. 12A shows a mood-adjusting system 1201 architecture diagram according to another embodiment. As shown in FIG. 12A, vital sign signals including biosignal data are obtained from the client subject 1221. Such signals may include signals obtained from iOximeter Vital 1231. Such vital sign signals are output either by hardware of wireless means to a vital sign database 1241, which is connected to vital signs and soundscape matching processor 1261, which provides soundscape and settings data to a soundscape catalog 1251. Vital sign database 1241, vital sign soundscape matching processor 1261, and soundscape catalog 1251 may be provided within a cloud 1271. The mood-adjusting system 1201 includes operator control panel computer system 1281 into which biosignal data, such as that obtained through vital sign sensors, is input. Soundscape and setting data is transmitted to operator control panel computer system 1281, and based thereon, soundscape signals are transmitted to speak system 1211, which generates an auditory stimulus based on the soundscape signals, which is tied in real-time to the measured physiological and psychology state of the client subject, as measured by the vital signs, and adjusts the mood of the client subject as desired.

FIG. 12B shows a mood-adjusting system 1201 architecture diagram according to another embodiment. Similar to the embodiment of FIG. 12A, client subject 1222 is provided with auditory stimulus by speaker system 1212. Vitals signs with biosignals are obtained from the client subject 1222 by an iOximeter Vital. The iOx is a vital signs monitoring hardware device that connects, for example, to smartphones through a headphone jack or USB-c connection. The iOX app processes the signal and produces the following output: Heart Rate (HR), Oxygen Saturation (SpO2). In this embodiment, the iOX pulse oximeter 1232 is used, but as noted, other vitals signs may be measured, including heart rate variability, heart rate, EEG, GSR, and oxygen saturation. The vital signs data are transmitted to a vital signs and soundscape record database 1262 through cloud 1272. The vitals signs data is transmitted to a computer system 1282 including a MaxMSP Visualizer, from which the vitals signs are transmitted to a processing device 1292 that performs a MaxMSP Biofeedback algorithm and process on the vital sign data, which generates the soundscape data, which is transmitted to the computer system 1282. Computer system with Max MSP Visualizer 1282 then transmits the soundscape signal to the speaker system, which in turn provides the auditory stimulus to the client subject 1222 based on the soundscape signal, which is tied in real-time to the measured physiological and psychology state of the client subject, as measured by the vital signs, and adjusts the mood of the client subject as desired.

According to this or other embodiments, the architecture of the system includes three primary components: (1) Max MSP Biofeedback processor that prepares and applies an algorithm, (2) an Orelo Demo App, and (3) Vital Signs+Soundscape Cloud Database. The system includes at least three secondary components that connect the primary components: (1) iOX MaxMSP Integration, (2) Vital Signs Export to Cloud, and (3) Soundscape Log Export to Cloud. It is again noted that although the MaxMSP platform is described in some embodiments, this disclosure is not limited to a MaxMSP platform or language. Other platforms or languages may equivalently and in fact may preferably be used, including but not limited to Pure Data (PD), AudioMulch, Bidule, Kyma, TouchDesigner, vvvv, OpenMusic, Nodal, and or other visual programming platforms.

Further, according to a further embodiment, a specialty version of an iOX app (APK or ipa file) is provided that has the ability to connect to a NodeJS service on a computer that is running MaxMSP that will receive the outputs from the iOX app. Additionally, a new variation of the iOX app is created with the above functionality but without the phone displaying the health results but instead a simplified interface for controlling the experience.

NodeJS is implemented for connection to Max MSP to export real-time data to the Max MSP patch. The Max MSP iOx patch connection allows the user to set the frequency of data updates from the iOX device (in Seconds). For example, a value of 4, will correspond to a measurement being updated every 4 seconds, with values allowed from 0.1 to 30 seconds.

FIG. 13 shows a schematic diagram of an environment of a relaxation inducing system 1300. A subject 1303 is placed in a comfortable situation, for example, in bed 1304, within a soundproof environment with soundproof walls 1340 and sound-absorbing structure 1345. High-fidelity speakers 1310, 1320 are provided on opposing sides of the subject 1303. A subwoofer 1335 is also provided. Additionally, a linear actuator may be provided such that haptic feedback as well as sound may be applied to the subject. The eyes of the subject may be covered with a light-blocking shield 1350. And a biosensor 1330, such as a pulse oximeter, is attached to the subject and obtains a biosignal from the subject that is correlated to a relaxed physiological response of the subject 1303. A resound box device 1315 may be provided that provides a high-quality audio signals to the respective speakers 1310, 1320 and subwoofer 1335 based on a soundscape signal transmitted to the box 1315.

According to this embodiment, the biosignal obtained from the biosensor 1330 is transmitted to a computer system 1360 through wire 1339 that performs processing on the obtained biosignal and based thereon, and provides a soundscape signal to be transmitted to the speakers 1310, 1320 to obtain a real-time adjustment to cause effective and efficient relaxation in the subject. Additionally, an operator 1370 may be provided with display screens 1360, 1365 to monitor the treatment of the subject. And a tablet 1380 is provided, which may transmit the biosignal and soundscape to a storage device in the cloud. Operator 1370, who may be a certified or trained therapist, may be at location near the subject being treated, or alternatively, the system may be arranged such that the operator 1370 may be remote from the subject, while receiving transmissions of the biosignals and applied audio stimuli in real time over a network, such as a local network or over the Internet. With such a system, the operator 1370 may adjust the applied audio stimuli to enhance the relaxation of the subject. And even still, the operator 1370 may receive stored biosignals and the corresponding applied audio stimuli such that the operator 1370 may provide audio stimuli at certain times based on certain biosignals to provide the subject such that a tailor-fit relaxation session.

In this embodiments, processing of the biosensor data obtained from the biosensor may be performed and generation of the adjusted soundscape signals may be performed by the computer system 1360. Or alternatively, the tablet 1380 or the computer system 1360, may transmit the biosensor data to a server or processor within a cloud network, and appropriate soundscape signals may be generated there and transmitted through the cloud network to the tablet 1380 or the computer system 1360, and from there, transmitted to, for example, the resound box 1315, and then transmitted to the respective speakers 1310, 1320 and subwoofer 1335.

According to this embodiment, the lighting level is adjustable. The bed 1304 is not a lie-flat bed, but rather a recline adjustable bed or chair, such as a lounge chair. If it is determined that the subject has fallen asleep, which in this case is not the desired object of the treatment, the subject may be woken through adjusting the lights or causing vibrations in the bed or chair. Additionally, olfactory stimulus associated with relaxation may also be applied to the subject, and tactile stimulus may also be applied, based, similar to the auditory stimulus, on the measured biosignal. Additionally, it is noted that hygiene is important in such an arrangement.

FIG. 14 provides another embodiment of the mood-adjusting system 1400, which may be arranged within the home of the subject 1403. The subject relaxes in bed or chair 1404. Preferably the subject's eyes are shielded by eye shield 1450. High-fidelity headphones 1410 are worn by the subject 1403, and a resound driver box device 1415 is arranged in proximity to the subject, which receives from the internet high quality 256-bit original digital recordings from the cloud that are used as the soundscape signal transmitted to the headphones. Biosignal data is obtained from biosensor 1430, which may be transmitted to a processor of a computer system in the cloud, for example, through wire 1439 and resound box 1415, which generates the soundscape signal based thereon, and transmits the soundscape signal to the resound box 1415.

A mobile version of a mood-adjusting, relaxation system 1500 is shown in FIG. 15. The subject 1503 lies on the beach on blanker 1504. A biosensor, such as those obtained through a smart watch 1530 as discussed above, transmits biosignal data to a mobile device, such as a mobile device 1560, such as a smart phone. The subject wears eye shield 1550 and high-fidelity headphones 1510. The mobile device 1560 and smart watch 1530 may communicate through Bluetooth or a wireless network 1595. In this embodiment, processing of the biosensor data may be performed and generation of the adjusted soundscape signals may be performed by the processor of mobile device 1560, such as a mobile phone or iPhone owned by the subject, on which an app is provided. Or alternatively, mobile device 1560, rather than process the biosensor data and generate the soundscape signals, may transmit the biosensor data to computer system and processor within a cloud network, and appropriate soundscape signals may be generated and transmitted by the computer system and processor is transmitted to the mobile device, which transmits the soundscape signal to the high-fidelity headphones. This embodiment would allow the maximum number of users to enjoy the benefits of the inventors' platform without having to purchase any specialized hardware.

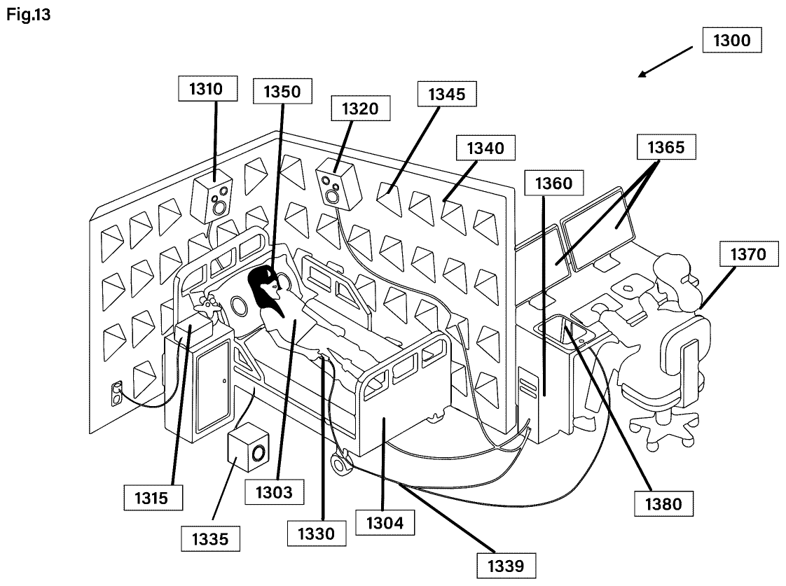

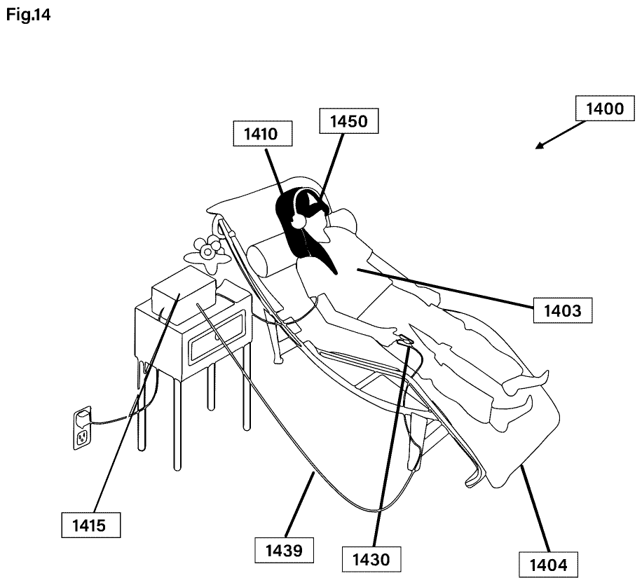

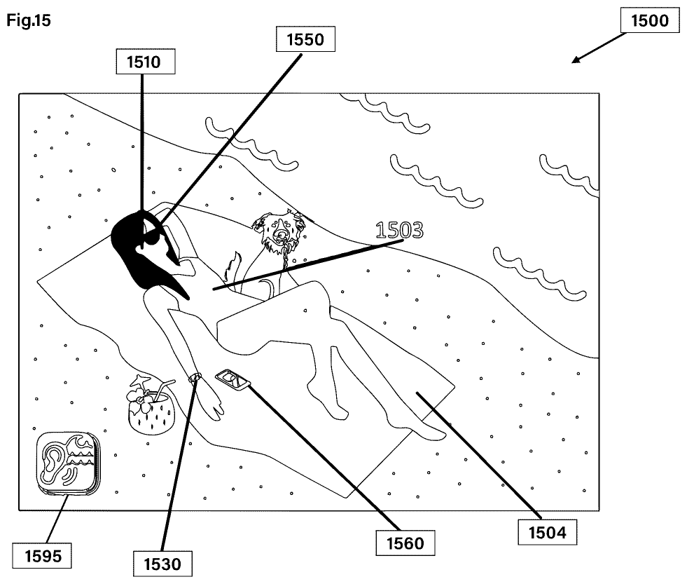

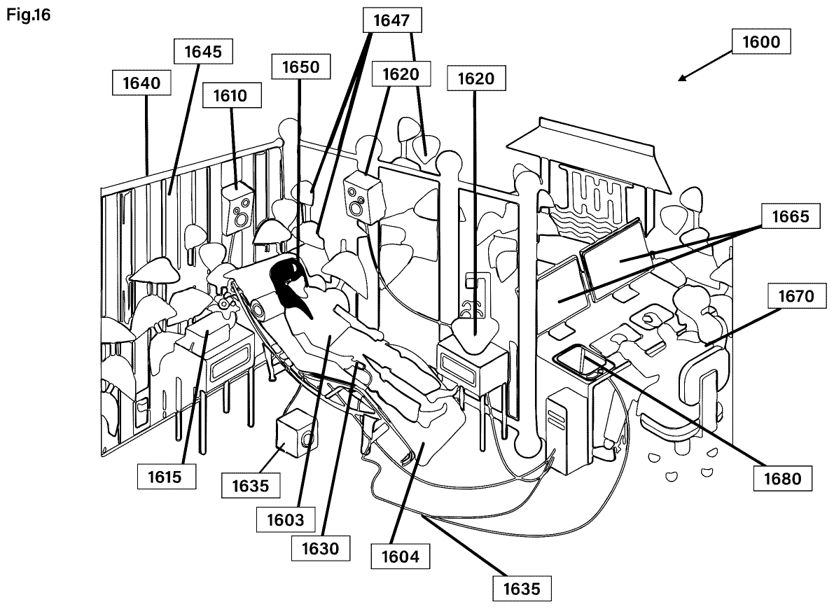

Lastly, a spa-type mood-adjusting, relaxation system 1600 is shown in FIG. 16. A subject 1603 is placed in a comfortable situation, for example, in bed or lounge chair 1604, within a soundproof environment with soundproof walls 1640 and sound-absorbing structure 1645. High-fidelity speakers 1610, 1620 are provided on opposing sides of the subject 1603. A subwoofer 1635 is also provided. Additionally, a linear actuator may be provided such that haptic feedback as well as sound may be applied to the subject. The eyes of the subject may be covered with a light-blocking shield 1650. And a biosensor 1630, such as a pulse oximeter, is attached to the subject and obtains a biosignal from the subject that is correlated to a relaxed physiological response of the subject 1603. A resound box device 1615 may be provided that provides a high-quality audio signals to the respective speakers 1610, 1620 and subwoofer 1635 based on a soundscape signal transmitted to the box 1615.

According to this embodiment, the biosignal obtained from the biosensor 1630 is transmitted to a computer system 1660 that performs processing on the obtained biosignal and based thereon, and provides a soundscape signal to be transmitted to the speakers 1610, 1620 to obtain a real-time adjustment to cause effective and efficient relaxation in the subject. Additionally, an operator 1670 may be provided with display screens 1665 to monitor the treatment of the subject. And a tablet 1680 is provided, which may transmit the biosignal and soundscape to a storage device in the cloud.

In this embodiments, processing of the biosensor data obtained from the biosensor may be performed and generation of the adjusted soundscape signals may be performed by the computer system 1660. Or alternatively, the tablet 1680 or the computer system 1660, may transmit the biosensor data to a server or processor within a cloud network, and appropriate soundscape signals may be generated there and transmitted through the cloud network to the tablet 1680 or the computer system 1660, and from there, transmitted to, for example, the resound box 1615, and then transmitted to the respective speakers 1610, 1620, and subwoofer 1635.

According to this embodiment, the lighting level is adjustable. The bed 1604 is not a lie-flat bed, but rather a recline adjustable bed or chair, such as a lounge chair. If it is determined that the subject has fallen asleep, which in this case is not the desired object of the treatment, the subject may be woken through adjusting the lights or causing vibrations in the bed or chair. Additionally, olfactory stimulus associated with relaxation may also be applied to the subject, and tactile stimulus may also be applied, based, similar to the auditory stimulus, on the measured biosignal. Additionally, it is noted that hygiene is important in such an arrangement.

According to the embodiment, which may be implemented in a spa environment or a yoga studio, the interior decoration includes of comfortable leather recliner, taffeta curtains, electric waterfalls, plants 1647, and other decorations designed to invoke a feeling of calm. Aromatherapy may also be implemented within the system, and the sound of flowing water may also be implemented to cause relaxation. The lounge chair 1604 may be a carbon fiber chair that arranges the subject such that the subject sits and the chair reclines so that knees and heart parallel to floor. Twinkling of artificial starts may also be provided on ceiling, and similar to the auditory stimulus, the twinkling color and frequency and beats of the twinkling may be driven by biosignals obtained from the subject such that the visual stimulus of even the starts is driven to an optimized efficient and effective relaxation of the subject.

In another embodiment, the mood-adjusting relaxation system is implemented in a portable booth that can be easily transported and set up in an outdoor space. The booth may be self-cleaning, such as a misting system, air filter, ultraviolet light, so that it is sterilized in between appointments. Visual indicators on the outside of booth can let the operator and customer know that it is in cleaning mode. A reservation system may be provided with the booth so that people are not waiting to try the experience. A staffer may also be provided to greet the customer and clean the booth.

FIG. 17 shows a soundscape production system 1700 for obtaining real-time biosignal data from a subject and generating soundscape signals to adjust a mood of a subject. The system 1700 includes computer system 1701, which may include a data input device 1710 configured to receive signals containing the biosignal data, a power source 1730, one or more processors 1735, a transmission module 1720, a user interface 1740, a communication module 1745, and one or more AI modules 1780.

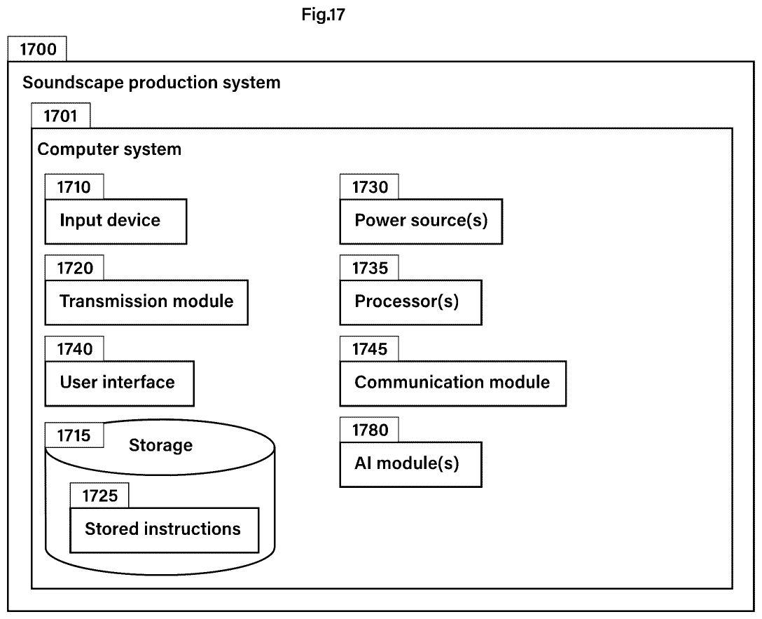

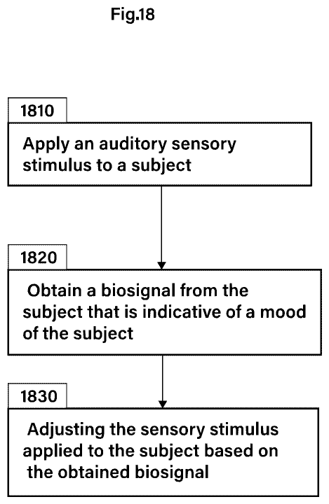

The storage 1715 may comprise instructions 1725 stored therein for operating a system for mood-adjusting stored thereon in a non-transitory form that, when executed by the one or more processors 1735, cause the one or more processors 1735 to carry out one or more of the steps described herein, in particular receiving the biosignal data and generating a soundscape signal to effective and efficiently adjust the relation state of the subject. The computer system 1701 may comprise one or more AI modules 1780 configured to apply the one or more neural networks. FIG. 18 shows steps of an embodiment of a method to adjust the mood of a subject. The steps include 1810 applying one or more sensory stimuli to a subject; 1820 obtaining one or more biosignals from the subject, the one or more biosignals being indicative or correlative of a mood of the subject; and 1830 adjusting the one or more sensory stimuli applied to the subject based on the obtained biosignals to adjust the subject to a predetermined mood, emotion, feeling, or affective state.

As described, the mood adjustment system and method may be implemented by an app provided on a subject's mobile device, such as a mobile phone or tablet. Because the system is related to relaxation, simple symbols on the mobile device are preferred to control the app. As shown in FIGS. 19A, 19B, and 19C, the simple icon 1900 for the app includes a circle 1910 and a horizontal bar 1920. The symbolism is a head resting on a pillow, on a sun setting over the horizon. This ambiguity is intentional. From the architecture, the interface is a replacement of the iOx interface so the core functionality is preserved. The iOx will connect via NodeJS to the MaxMSP setup.

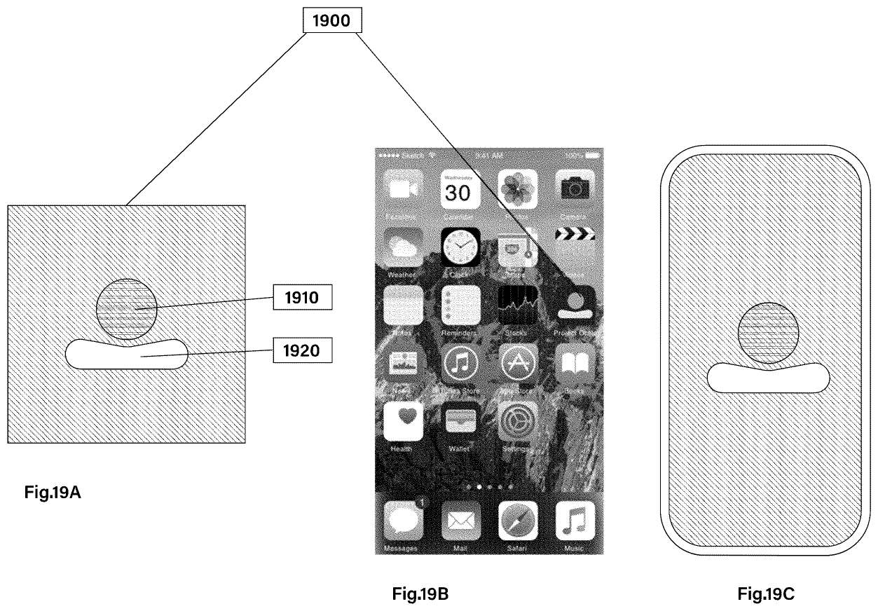

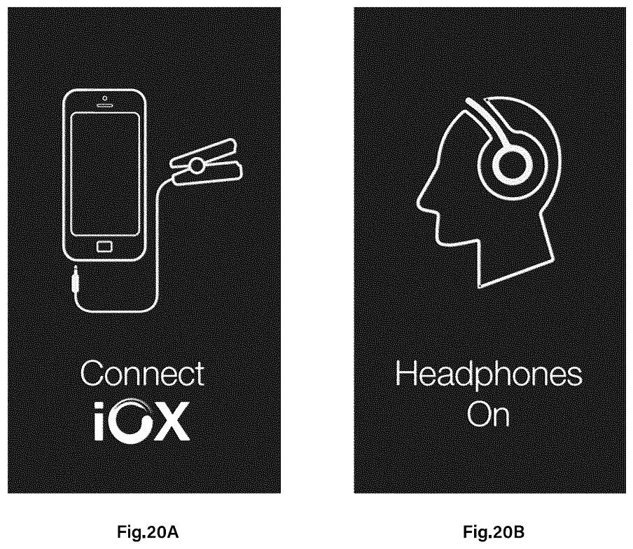

Upon the initial startup of the app, the app shows the head (the yellow circle) laying on the pillow (the horizontal bar). An animation moves the head to the Start position and the background changes color as well. FIG. 20A shows the app instructions for the subject to connect the iOX. The session cannot proceed without the iOx plugged into the phone. The app further displays FIG. 20B showing headphones to remind the user to put their headphones on.

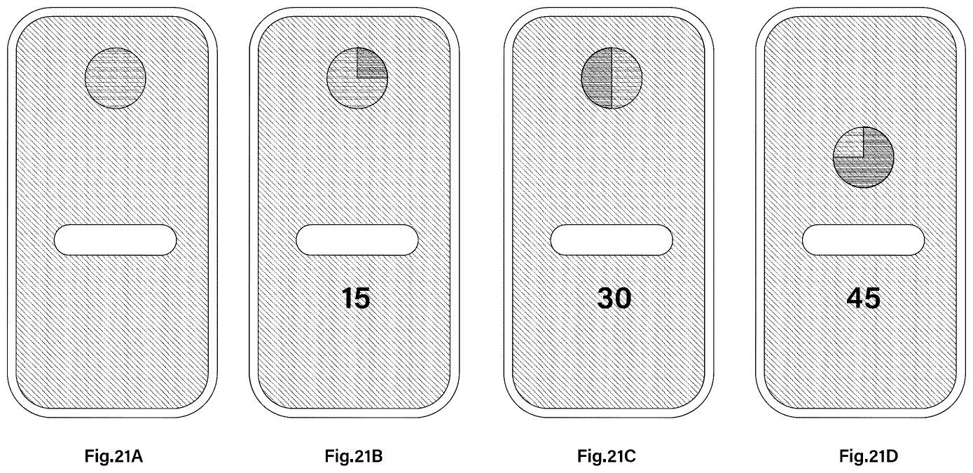

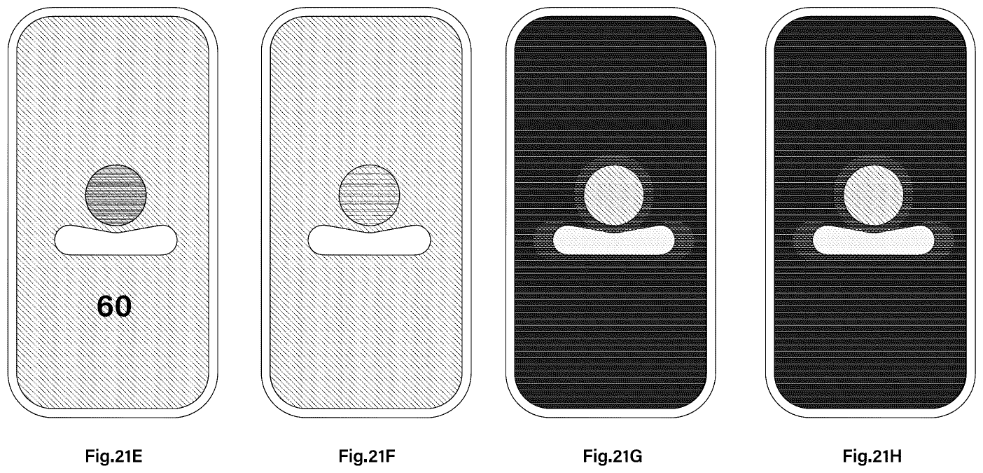

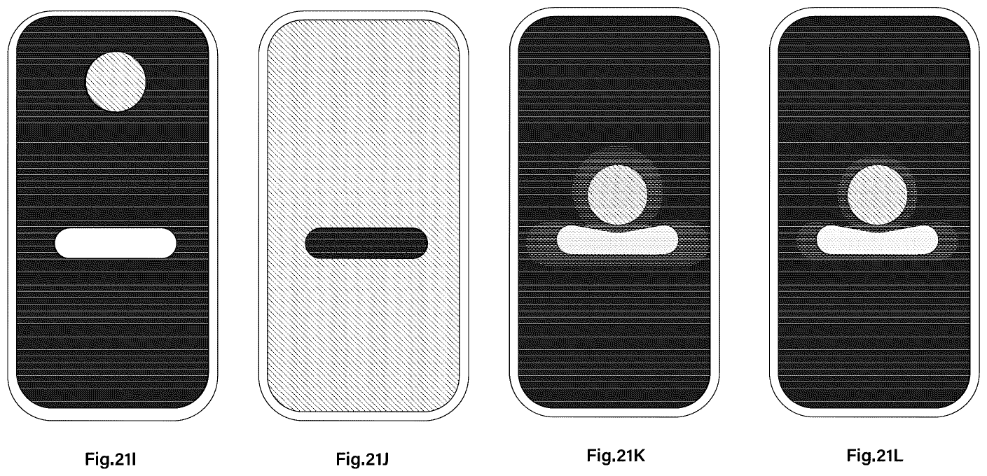

As shown in FIG. 21A, on the initial screen, the user begins by pressing the circle and holds it down, dragging the timer to the correct position and letting go. FIG. 21B shows a timer, which is the first position. The user can press and drag the head down to different positions representing 30, 45, and 60 minutes, which may be the selected duration of the session. FIG. 21C shows a timer for 30. For each time segment, the number that corresponds to the timer setting displays beneath the pillow. Also, a corresponding slice of the head is shaded. FIG. 21D shows a timer for minutes. To start the session, the user just needs to click on the head to begin, and the session will count down from the number of minutes selected. FIG. 21E shows a timer with 60 minutes, which is the maximum amount of time per session. FIG. 21F shows a start of the application. Once the user lets go of the head, no matter where it is, an animation shows that it drops below and rests on the pillow and the session begins. FIG. 21G shows the app is in session. Once the user lets go of the head, the background will go to the dark color and the head and pillow will display a glowing aura to denote that the session has started. FIG. 21H shows the remaining time in the session. If this is a timed session, then there will be a display below the pillow with the number of minutes remaining in the session. FIG. 21I shows a pause for the app. During the session, if the user wants to pause the experience, they can hold down the circle for 2 seconds to pause. When this pauses, the head lifts back up from the pillow. If the user wants to stop altogether, they hold down the pillow for 2 seconds. FIG. 21J shows an end of the session. Once the session is over, the head rises from the pillow and leaves the screen. This lets the user know that the session is complete. If the user wants to experience another session, they can press the pillow and the head will come back to the position in the Opening screen. FIG. 21K shows a volume up when the volume is increased during or before the session begins. Pressing two fingers to the screen and sliding upward will raise the volume. And FIG. 21L shows a volume down when the volume is decreased during or before the session begins. Pressing two fingers to the screen and sliding downward will lower the volume. With the icons and interface described herein, the initiation and control of the session is simplified in a way that lends itself well to the relaxation that the session is intended to induce.

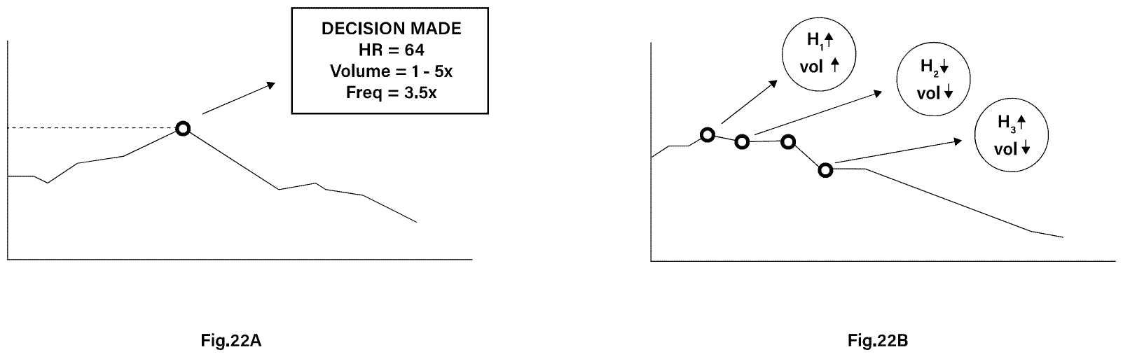

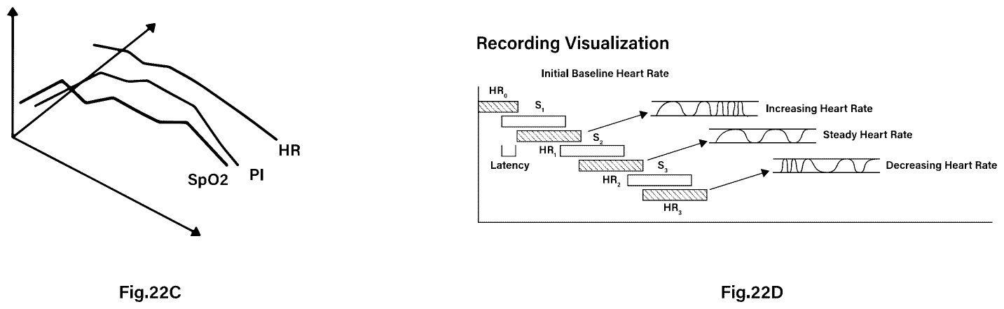

As described above, the method and system described herein are intended to provide for mood adjusting of the subject, such as relaxation of the subject, based on biosensor data obtained from the subject. In the embodiments directed to relaxation of the subject by applied auditory soundscapes, various aspects of the applied auditory soundscape are adjusted based on real-time biosensor data obtained from the subject to most effectively produce the desired physiological or psychological response or physiological or psychological changes in the subject. FIGS. 22A, 22B, 22C, and 22D show examples of how data are analyzed to determine how the auditory signal should be changed or maintained based on the real-time biosensor data. For example, in FIG. 22A, a decision is based on the heart rate being at a certain location (HR=64), and the applied soundscape being at a certain volume and a certain frequency being applied to the subject. FIGS. 22C and 22D show a visualization of data obtained from the biosignal. FIG. 22D particularly shows two lines, HR and Spo2 with x axis being time and y axis being the values. Callouts are shown at each decision point showing the inputs and why the audio engine made the decision. The algorithm on which the soundscape signals are generated may further look to predetermined thresholds reached within the data, predetermined threshold in which the measured signals remains over a specified period of time, an amount of change of measured biosignals over time, a comparison of multiple biosignals, or first or second derivatives of the measured biosignals.

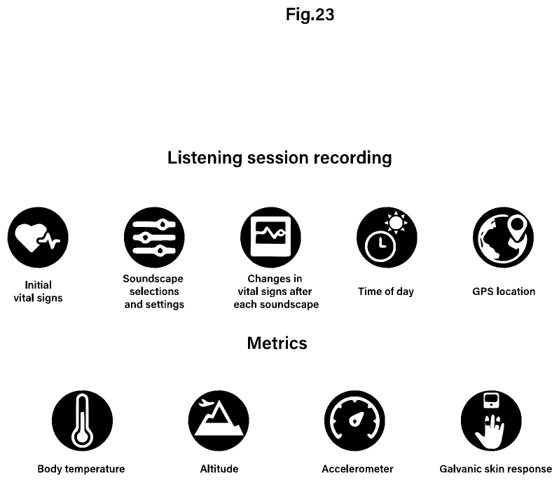

In the embodiments described herein, the biosignals data may include sound/heart rate latency. Decision points may be based on heart rate data. As shown in FIG. 23, GPS data from the phone of the subject may also be considered in generating the soundscape signal, body temperature, time of day, altitude, accelerometer data from the subject, and galvanic skin response (GSR) may also be used in the metrics in generating the soundscape signal.

According to an embodiment of the invention, one of the two leading fitness trackers on the market may be used: Fitbit and Apple Watch to obtain the biosignals. Apple Watch does allow continuous measurement of the heartrate without the user going to Exercise mode. The advantages of the Apple Watch are that the SDK is consistent across all devices. A downside with Apple Watch is that it limits the integration to the Apple family of products and does not support Fitbit. Fitbit has two types of products—the Charge series which upload the data to the cloud, and the devices that run the Fitbit OS. They are different types of devices with the Fitbit OS devices being more sophisticated technically. Only the Fitbit Ionic and Versa models, which run the Fitbit OS have SDKs allow creation of 3rd-party applications that run on them. Even though Fitbit is the number-one fitness tracker, there is a smaller percentage of users of Fitbit who have the Fitbit Versa rather than one of the devices that cannot be integrated.

Bluetooth 5.0 Wireless headphones may be used as the high-fidelity headphones. Headphone with an oximeter, accelerometer, galvanic skin response, body temperature may be used. The oximeter emitter and sensor may be placed on the foam cup of one ear. The accelerometer maybe provided in the headband. A GSR sensor may include a metal strip on top of the cushion of the headphones. The body temperature may include an infrared ear sensor. The audio driver should preferably be at least as good as Monoprice Premium DJ Headphones. Alternatively, an EEG may be incorporated into the headphones. Alternatively, glasses with biosensors may be worn by the subject.

It is significant to note that soundscape generation is based on biofeedback. And although many of the above embodiments are directed to relaxation of the subject, the same inventive principles may be applied to other moods, such as increase energy level, euphoria, cravings, excitement, wakefulness, motivation to endure, anxiety, or even an increased libido or sexual desire. The soundscape signal is generated dynamically based on the physiological reading. All audio signals are preferably generated in real-time according to bio signals, such as GSR, EEG, pulse, oxygen saturation, respiration rate, etc. Remote diagnosis can be carried out through the Internet. The measurement data of human body characteristic signals will be sent to a cloud server.

The server will recognize the machine learning algorithm [or with a human interpretation and manual guidance] and give the treatment plan. The treatment plan will include various physical interventions such as audio frequency intervention and light wave intervention.

According to embodiments of the disclosed method and system, soundscape settings are automatically generated by a computer system through algorithms via biofeedback sensors and delivers a personalized relaxation experience to the user. The system learns from previous sessions to enhance the user's experience. The system may be based on smart watches, electroencephalograms (EEG), galvanic skin response (GSR) sensors, and pulse oximeters.

Embodiments of the present disclosure may comprise or utilize a special-purpose or general-purpose computer system that includes computer hardware, such as, for example, one or more processors and system memory, as discussed in greater detail below. Embodiments within the scope of the present disclosure also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

Certain terms are used throughout the description and claims to refer to particular methods, features, or components. As those having ordinary skill in the art will appreciate, different persons may refer to the same methods, features, or components by different names. This disclosure does not intend to distinguish between methods, features, or components that differ in name but not function. The figures are not necessarily drawn to scale. Certain features and components herein may be shown in exaggerated scale or in somewhat schematic form and some details of conventional elements may not be shown or described in interest of clarity and conciseness.

Although various example embodiments have been described in detail herein, those skilled in the art will readily appreciate in view of the present disclosure that many modifications are possible in the example embodiments without materially departing from the concepts of present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features from the various embodiments disclosed may be employed in combination. In addition, other embodiments of the present disclosure may also be devised which lie within the scopes of the disclosure and the appended claims. Each addition, deletion, and modification to the embodiments that falls within the meaning and scope of the claims is to be embraced by the claims.

Certain embodiments and features may have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges may appear in one or more claims below. Any numerical value is “about” or “approximately” the indicated value, and takes into account experimental error and variations that would be expected by a person having ordinary skill in the art.

1. A system for adjusting a mood of a subject, the system comprising:
a sensory stimulator system configured to apply one or more sensory stimuli to a subject;
a sensor system configured to obtain one or more biosignals from the subject, the one or more biosignals being indicative or correlative of a mood of the subject; and
a computer system having one or more processors configured to receive the one or more obtained biosignals, and based thereon, generate a stimuli signal to adjust the sensory stimuli applied to the subject by the sensory stimulator system;
wherein the sensory stimulator system adjusts the one or more sensory stimuli applied to the subject based on the generated stimuli signal to obtain a predetermined mood, emotion, feeling, or affective state in the subject.

2. The system according to claim 1, wherein the sensor system includes one or more of sensors configured to obtain from the subject data relating to electrodermal activity (EDA), galvanic skin response (GSR), electrodermal response (EDR), psychogalvanic reflex (PGR), skin conductance response (SCR), sympathetic skin response (SSR) and skin conductance level (SCL), blood pressure (BP), pulse oximetry, oxygen saturation, electroencephalography (EEG), electromyography (EMG), body movement based on one or more accelerometers or one or more gyroscopes, electrocardiography (ECG), temperature of the subject, thermal imaging, respiration, visual images of the subject, heart rate (HR), heart rate variability (HRV), photoelectric plethysmography (PPG), photoplethysmography imaging (PPGI), prefrontal cortex activity, oxyhemoglobin (oxy-Hb) concentration, cortisol levels including salivary cortisol levels, hair cortisol levels, and/or fingernail cortisol levels, pupil dilation, pupillometry, pulsimetry, accelerated plethysmography (APG), optical imaging of tissues of the subject including functional near infrared spectroscopy (fNIRS), functional magnetic resonance imaging (fMRI), computed tomography (CT), magnetoencephalography (MEG), positron emission tomography (PET), or infrared spectroscopy (NIRS).

3. The system according to claim 1, wherein the sensory stimulator system is configured to apply auditory stimuli, visual stimuli, tactile stimuli, olfactory stimuli to the subject, or taste-based stimuli to the subject.

4. The system according to claim 1, wherein the sensory stimulator system is an auditory system configured to apply auditory stimuli that includes providing one or more of the following: introductory music, a frequency scan of sound of varying frequencies, heartbeat-mimicking sound, instrumental music, natural sounds, and/or binaural beats.

5. The system according to claim 1, wherein the sensory stimulator system is an auditory system configured to apply auditory stimuli that includes providing one or more of the following sounds in consecutive sequence: introductory music, a frequency scan of sound of varying frequencies, heartbeat mimicking sound, instrumental music, natural sounds, binaural beats, and/or closing music.

6. The system according to claim 1, wherein the sensory stimulator system is an auditory system configured to apply auditory stimuli that includes providing one or more of the following sounds concurrently: introductory music, a frequency scan of sound of varying frequencies, heartbeat mimicking sound, instrumental music, natural sounds, binaural beats, and/or closing music

7. The system according to claim 1, wherein the sensory stimulator system is an auditory system configured to apply auditory stimuli that includes providing a frequency scan of sound of varying frequencies including at least sound of a first frequency and sound of a second frequency,
providing a frequency scan of sound of varying timbres including at least sound of a first timbre and sound of a second timbre,
providing a frequency scan of sound of varying harmonies including at least sound of a first harmony and sound of a second harmony,
providing a frequency scan of sound of varying loudness including at least sound of a first loudness and sound of a second loudness,
a frequency scan of sound of varying pitch including at least sound of a first pitch and sound of a second pitch,
providing a frequency scan of sound of varying tones including at least sound of a first tone and sound of a second tone, or
providing a frequency scan of sound of varying pure tones including at least sound of a first pure tone and sound of a second pure tone.

8. The system according to claim 1, wherein the sensory stimulator system is an auditory system configured to apply auditory stimuli that includes
introductory music
a heartbeat mimicking sound
instrumental music
natural sounds, or
a reproduction of man or machine-made-sounds.

9. The system according to claim 1, wherein the sensory stimulator system is a visual system configured to apply visual stimuli that includes providing visual light of varying frequencies, brightness, pulses, or combinations thereof or in varying patterns.

10. The system according to claim 1, wherein the sensory stimulator system is a tactile system configured to apply tactile stimuli that includes varying force or pressure, providing vibrations of varying frequencies and varying amplitudes and to different parts of the subject, and/or applying varying temperatures to different parts of the subject.

11. A method for adjusting a mood of a subject, the method comprising:
applying one or more sensory stimuli to a subject;
obtaining one or more biosignals from the subject, the one or more biosignals being indicative or correlative of a mood of the subject;
receiving the one or more obtained biosignals and processing said biosignals by a computer system having one or more processors and based thereon, generating a stimuli signal to adjust the sensory stimuli applied to the subject; and
adjusting the one or more sensory stimuli applied to the subject based on the generated stimuli signal to obtain a predetermined mood, emotion, feeling, or affective state in the subject.

12. The method according to claim 11, wherein the one or more biosignals obtained from the subject include data relating to electrodermal activity (EDA), galvanic skin response (GSR), electrodermal response (EDR), psychogalvanic reflex (PGR), skin conductance response (SCR), sympathetic skin response (SSR) and skin conductance level (SCL), blood pressure (BP), pulse oximetry, oxygen saturation, electroencephalography (EEG), electromyography (EMG), body movement based on one or more accelerometers or one or more gyroscopes, electrocardiography (ECG), temperature of the subject, thermal imaging, respiration, visual images of the subject, heart rate (HR), heart rate variability (HRV), photoelectric plethysmography (PPG), photoplethysmography imaging (PPGI), prefrontal cortex activity, oxyhemoglobin (oxy-Hb) concentration, cortisol levels including salivary cortisol levels, hair cortisol levels, and/or fingernail cortisol levels, pupil dilation, pupillometry, pulsimetry, accelerated plethysmography (APG), optical imaging of tissues of the subject including functional near infrared spectroscopy (fNIRS), functional magnetic resonance imaging (fMRI), computed tomography (CT), magnetoencephalography (MEG), positron emission tomography (PET), or infrared spectroscopy (NIRS).

13. The method according to claim 11, wherein the one or more biosignals are obtained by retrieving the biosignals from a data storage having the data of the biosignal previously stored thereon or by receiving data of the biosignals.

14. The method according to claim 11, wherein applying the one or more sensory stimuli includes applying auditory stimuli, visual stimuli, tactile stimuli, olfactory stimuli to the subject, or taste-based stimuli to the subject.

15. The method according to claim 11, wherein applying the one or more sensory stimuli includes applying auditory stimuli that includes providing one or more of the following: introductory music, a frequency scan of sound of varying frequencies, heartbeat-mimicking sound, instrumental music, natural sounds, and/or binaural beats.

16. The method according to claim 11, wherein applying the one or more sensory stimuli includes applying auditory stimuli that includes providing one or more of the following sounds in consecutive sequence or concurrently: introductory music, a frequency scan of sound of varying frequencies, heartbeat mimicking sound, instrumental music, natural sounds, binaural beats, and/or closing music.

17. The method according to claim 11, wherein applying the one or more sensory stimuli includes applying auditory stimuli that includes providing a frequency scan of sound of varying frequencies including at least sound of a first frequency and sound of a second frequency,
providing a frequency scan of sound of varying timbres including at least sound of a first timbre and sound of a second timbre,
providing a frequency scan of sound of varying harmonies including at least sound of a first harmony and sound of a second harmony,
providing a frequency scan of sound of varying loudness including at least sound of a first loudness and sound of a second loudness,
a frequency scan of sound of varying pitch including at least sound of a first pitch and sound of a second pitch,
providing a frequency scan of sound of varying tones including at least sound of a first tone and sound of a second tone, or
providing a frequency scan of sound of varying pure tones including at least sound of a first pure tone and sound of a second pure tone.

18. The method according to claim 11, wherein applying the one or more sensory stimuli includes applying auditory stimuli that includes
introductory music
a heartbeat mimicking sound
instrumental music
natural sounds,
a reproduction of man-made-sounds, or
a reproduction of man-made-sounds or of machine-made-sounds.

19. The method according to claim 11, wherein the sensory stimulator system is
a visual system configured to apply visual stimuli that includes providing visual light of varying frequencies, brightness, pulses, or combinations thereof or in varying patterns, or
a tactile system configured to apply tactile stimuli that includes varying force or pressure, providing vibrations of varying frequencies and varying amplitudes and to different parts of the subject, and/or applying varying temperatures to different parts of the subject.

20. A hardware storage device having stored thereon computer executable instructions which, when executed by one or more processors of a computer system, configure the computer system to perform at least the following:
apply one or more sensory stimuli to a subject;
obtain one or more biosignals from the subject, the one or more biosignals being indicative or correlative of a mood of the subject;
receive the one or more obtained biosignals and processing said biosignals by a computer system having one or more processors and based thereon, generating a stimuli signal to adjust the sensory stimuli applied to the subject; and
adjust the one or more sensory stimuli applied to the subject based on the generated stimuli signal to obtain a predetermined mood, emotion, feeling, or affective state in the subject.
